(19) United States
(12) Patent Application Publication
(10) Pub. No.: US 2005/0171541 A1
(43) Pub. Date: Aug. 4, 2005

(54) Device for lumbar surgery

(76) Inventors: Frank H. Boehm JR., Utica, NY (US); Benedetta B. Melnick, Rome, NY (US)

Correspondence Address: DILWORTH & BARRESE, LLP, 333 EARLE OVINGTON BLVD., UNIONDALE, NY, 11553, US

(21) Appl. No.: 11/018,610

(22) Filed: Dec. 20, 2004

Related U.S. Application Data
(60) Provisional application No. 60/531,389, filed on Dec. 19, 2003.

(51) Int. Cl. A61B 17/70

(52) U.S. Cl. 606/61

(57) ABSTRACT

A method for performing percutaneous interbody fusion is disclosed. The method includes the steps of inserting a guide needle posteriorly to the disc space, inserting a dilator having an inner diameter slightly larger than the outer diameter of the guide needle over the guide needle to the disc space to enlarge the disc space, and successively passing a series of dilators, each having an inner diameter slightly larger than the outer diameter of the previous dilator, over the previous dilator to the disc space the gradually and incrementally increase the height of the disc space. Once the desired disc height is achieved, the guide needle and all the dilators, with the exception of the outermost dilator, are removed. An expandible intervertebral disc spacer is then passed through the remaining dilator and positioned in the disc space. The disc spacer is expanded to the required disc height, and then a bone matrix is passed through the dilator to fill the disc space. The dilator is then removed. An expandible intervertebral disc spacer is also disclosed, having a tapered bore that causes greater expansion of one end of the spacer with respect to the other. A kit for performing the percutaneous interbody fusion procedure is also disclosed.

### CROSS-REFERENCE TO RELATED APPLICATION AND CLAIM FOR PRIORITY

This application claims priority to U.S. Application No. 60/531,389 entitled “Device and Method for Lumbar Surgery,” filed on May 4, 2004.

### BACKGROUND

1. Field of the Invention

The present invention relates to a device and method for performing interbody spinal fusion, stabilization and restoration of the disc height in the spine, and in particular to a device and method for performing percutaneous, minimally invasive interbody fusion of the lumbar spine.

2. Discussion of the Related Art

Many devices exist to assist in maintaining the position of the lumbar vertebra in conjunction with lumbar fusion surgery. Fusion is the joining together of the vertebra of the spine. The underlying concept of the known devices is to maintain the relative position of the vertebral bodies with respect to each other, while the bone that has been placed between the vertebra to form the fusion of the vertebra, has an opportunity to heal and mature. These devices employ different strategies and philosophies, and can include devices which utilize the pedicles, as well as devices which are placed in to the disc space to promote fusion across the disc space. The latter devices and techniques associated with these devices are known as “interbody fusion”. While no single technique has been universally accepted as the most optimum method, there is growing evidence that interbody fusion may be the preferred method.

The interbody fusion procedure may be performed via an anterior or posterior approach. Initially, all interbody fusion procedures were accomplished using the posterior approach. The procedure was performed by first performing a laminectomy, removing the disc space, and then packing the disc space with pieces of bone, which were then permitted to heal over time. The hope was that the inserted bone pieces would grow and fuse together with the vertebra above and below that disc space, forming a bridge of bone between the two vertebral bodies, thus accomplishing the interbody fusion.

Posterior interbody fusion procedures are accomplished via a variety of techniques. Most procedures attempt to restore proper disc height, i.e. the space between the adjacent vertebra. The patient benefits from restoring the proper disc height, particularly where there has been deterioration, degeneration or collapse of the disc.

More recently, the anterior interbody fusion procedure has gained popularity, due to the availability and improvements made in devices that enable the anterior approach for lumbar interbody fusions. These devices typically provide for a retroperitoneal or transperitoneal technique to be used for approaching the lumbar disc, removing some or all of the disc, and placing either bone or a metallic device into the disc space. These devices also typically provide a means for distracting the disc space, i.e. making the space between the discs wider. Presently, this aspect of lumbar interbody fusion procedures are considered to be an important step in the procedure because of its effects on the neural foramina, or areas from which the nerve roots exit through the vertebra. It is generally accepted that enlarging the disc space consequently enlarges the neural foramina, thus decompressing the exiting nerve roots.

The current techniques, due to the present equipment available, particularly for anterior interbody fusion, suffer the disadvantage in that they are major surgeries and require large incisions with the manipulation of both tissue and organs. While attempts have been made to perform anterior interbody fusions laparoscopically, these procedures are often complicated and are typically performed under general anesthesia.

Therefore, a need exists for a method for performing interbody fusions that reduces the trauma to the patient, and consequently reducing recovery time. A device is also needed to facilitate the interbody fusion procedure to enable the procedure to be performed percutaneously, enabling the surgeon to distract the disc to restore disc height, maintain the distraction, and promote the growth of the bone placed in the disc space between the two vertebral bodies, thus accomplishing the interbody fusion.

### SUMMARY OF THE INVENTION

It is, therefore, an object of the present invention to provide a minimally invasive, percutaneous surgical procedure for performing interbody fusion which reduces the trauma to the patient and reduces recovery time.

It is also an object of the present invention to provide a percutaneous interbody fusion procedure which accomplishes the interbody fusion through small incisions in the body of the patient and utilizes a minimum of incisions to complete the procedure.

It is a further object of the present invention to provide a device which facilitates the percutaneous interbody fusion procedure.

It is yet another object of the present invention to provide a device which distracts the disc space and which may be inserted through a tube to effect the percutaneous interbody fusion procedure.

It is a furtHer object of the present invention to provide a collapsible and expandible interbody fusion spacing device that facilitates the percutaneous interbody fusion procedure.

It is still a further object of the present invention to provide a kit for performing a minimally invasive percutaneous interbody fusion procedure.

The above and other objects of the present invention may be achieved by providing a collapsible and expandible interbody fusion spacer device that may be inserted through a small diameter tube to the disc space that is being fused, so that the procedure may be performed in a minimally invasive manner. The spacer is preferably constructed in two halves that are connected by pins located on the sides of the spacer. The outer surface may be flat to engage the end plate of the vertebra above and below the spacer, and the outer surface may be scored, have ridges, points, tabs, detents, or the like to enhance gripping of the end plates of the vertebra to resist movement of the spacer once it is in place. The interior surfaces of the halves that make up the spacer include a semicircular hollowed portion that is preferably threaded along at least a portion of its length that is aligned with a similar semicircular threaded hollowed portion on the other half of the spacer. When the spacer is assembled, the threaded portion forms a canal for acceptance of a piston screw. Preferably, the threaded canal is tapered from one end to the other, particularly from the end which will be positioned posteriorly in the disc space to the end which will be positioned anteriorly in the disc space. When the piston screw is inserted, the anteriorly positioned end will expand a greater distance in the disc space than the posterior end, due to the tapered threaded canal. This will cause the disc height, i.e. the distance between the vertebra, to be greater anteriorly than posteriorly, which more closely mimics the natural curve of the spine, particularly in the lumbar spine, thus restoring lordosis, the natural curve of the lumbar spine.

A method for performing percutaneous interbody fusion is also provided, in which the disc space is enlarged in the craniocaudal direction following percutaneous discectomy. Following the discectomy, a guide needle is passed through the incision to the disc space between the vertebra. Over the needle, a series of tubularly shaped dilators are passed, with each successive dilator having an inner diameter that is slightly larger than the outer diameter of the dilator that is in place. As each successive dilator is inserted in the disc space, it forces the vertebra apart, increasing the disc space, until a desired height between the vertebra is achieved. Once a desired height is reached, which is only a desired height and not necessarily the maximum height, the outer dilator is left in place, while those inside the outer dilator are removed. The maximum height does not have to be achieved by the dilators because the expandible intervertebral disc spacer of the present invention is then inserted into the disc space through the outer dilator. Once in place, the spacer is expanded to increase the disc height to the maximum distance. After the spacer is in place on one side of the vertebral body, the procedure is repeated on the other side. After the two spacers are in place, a bone matrix, which encourages fusion, is passed through the dilators, filling the space with bone. The dilators are then removed and the procedure is complete.

A kit for performing percutaneous interbody fusion is also provided, which includes a plurality of expandible intervertebral disc spacers, which preferably expand the disc space a greater distance anteriorly than posteriorly, at least one dilator for expanding the disc height and having a hollow interior for allowing passage of the disc spacers to the disc space, and a guide needle. A curette for performing percutaneous discectomies may be provided, and a bone matrix for fusing the vertebra together may also be provided.

### BRIEF DESCRIPTION OF THE DRAWINGS

The above and other objects, features and advantages of the present invention will become more readily apparent from the following detailed description of preferred embodiments of the invention, taken in conjunction with the accompanying drawings, in which:

FIG. 1 illustrates a perspective view of an expandible intervertebral disc spacer according to the present invention;

FIG. 2 illustrates a side cross-sectional view of the disc spacer of FIG. 1;

FIG. 3 illustrates a perspective view of an alternative embodiment of the expandible intervertebral disc spacer of FIG. 1;

FIG. 4 illustrates a side cross-sectional view of the disc spacer of FIG. 3;

FIG. 5 illustrates diagrammatic view of a dilator system for enlarging the disc height of the vertebra prior to placement of the disc spacer of the present invention between the vertebra;

FIG. 6 illustrates a diagrammatic view of the placement procedure of the disc spacer of the present invention;

FIG. 7 illustrates a diagrammatic view of the disc spacer in place between the vertebra and in a fully expanded condition to restore the natural curvature of the spine;

FIG. 8 illustrates a percutaneous interbody fusion kit according to the present invention;

FIG. 9 illustrates a flow chart of the percutaneous interbody fusion method according to the present invention;

FIG. 10 is a front view of a disc spacer according to an alternative embodiment of the present invention; and

FIG. 11 is a front view of the disc spacer in FIG. 10 with an expander inserted therein.

### DETAILED DESCRIPTION

Referring now to the drawings, in which like reference numerals identify similar or identical elements throughout the several views, and inn particular to FIG. 1, there is shown the expandible intervertebral disc spacer device 10 according to the present invention. Preferably, the disc spacer 10 is comprised of two similarly shaped halves 12, 14 that are opposed to each other and loosely connected by pins 16. The outer surface of each half may be scored, as indicted by reference numeral 22, for facilitating adherence to the end plates of the vertebral bodies between which disc spacer 10 is placed. When top half 12 and bottom half 14 are assembled, together they may form a cylinder, a cube, a rectangular box, or any geometric shape that may be split to form two opposed halves. A tapered bore 18 is provided, which has a larger diameter 30 at a first end and a smaller diameter 32 at a second end. Preferably, tapered bore 18 is threaded over at least a portion of its length. While disc spacer 10 is preferably constructed of titanium or other suitable metal alloy, cortical bone may also be used. It is also contemplated that the material of the disc spacer 10, or at least the material of which tapered bore 18 is constructed, is self-tapping so that threads are not needed.

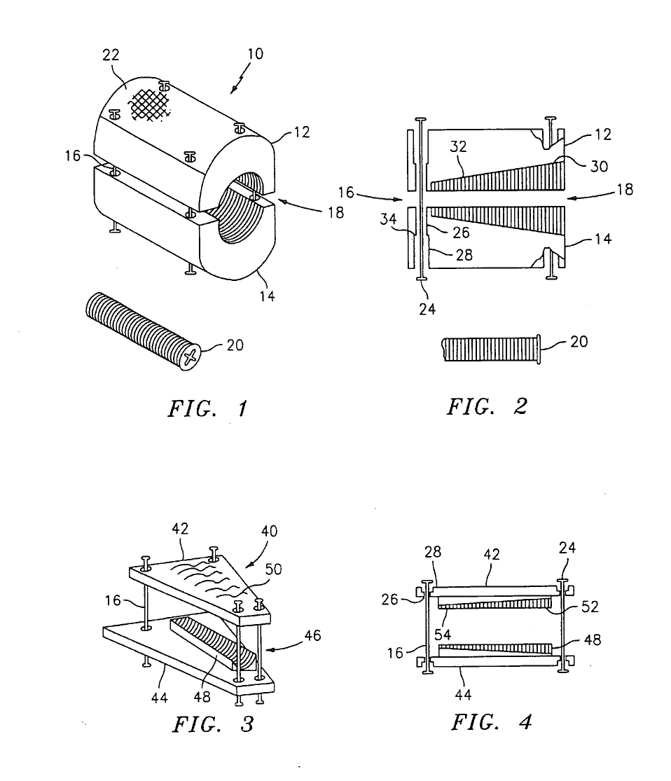

Screw 20 is provided for insertion into bore 18 to expand the disc spacer 10. As seen in FIG. 2, pins 16 are located in pin bores 26 which have a larger diameter near the outer surface of disc spacer 10, and a smaller diameter near the interior of the spacer. The change in diameter creates a stop 34 which engages the head 24 of pins 16, to terminate expansion of the spacer 10. When screw 20 is inserted into bore 18, the smaller diameter 32 of the threaded bore causes a greater expansion at the second end than at the first, for reasons which will be described below.

FIGS. 3 and 4 illustrate an alternative embodiment of the disc spacer 40 of the present invention. Disc spacer 40 comprises a pair of opposed plates 42, 44 which may be square, rectangular, rhomboidal, trapezoidal, or any suitable geometric shape. Pins 16 loosely hold the plates together, as described above, through pin bores 26, which include larger diameter portion 28 which creates stop 34 to engage the head 24 of pins 16. The outer surface of plates 42, 44 may include ridges 50, detents, scoring or the like to enhance adherence to the end plates of the vertebra. Each plate includes a threaded ledge portion 48, which forms a bore for accepting screw 20 when the plates are assembled to form disc spacer 40. Preferably, the threaded portion has a larger diameter at a first end 52 and a smaller diameter at a second end 54, so that there is greater expansion of the spacer at the second end 54 than at first end 52, for reasons which will be described below.

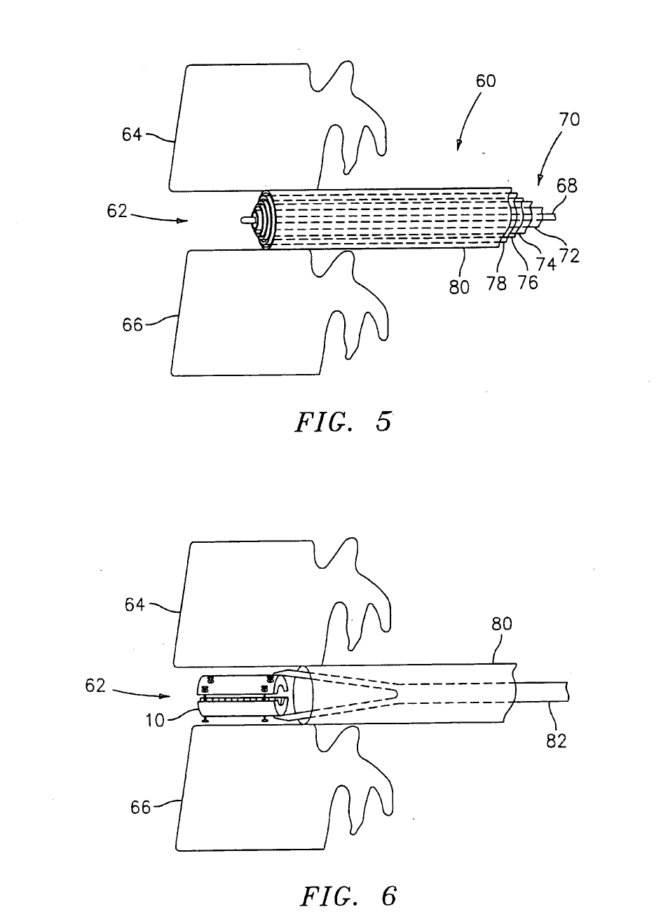

FIGS. 5, 6 and 7 illustrate the percutaneous interbody fusion procedure of the present invention, utilizing the expandible intervertebral disc spacer of the present invention. Following a percutaneous discectomy in which the disc between vertebra 64 and 66 is removed, preferably posteriorly, through a small incision, disc space 62 is enlarged using dilator system 60 in the procedure according to the present invention. In the procedure, a guide needle 68 is inserted into the disc space under scanning imaging, preferably fluoroscopy. Once the guide needle 68 is in place in the disc space 62, a series of dilators 70 are inserted over guide needle to enlarge the disc space. A first dilator 72, having an inner diameter that is slightly larger than the outer diameter of guide needle 68 is passed over the guide needle through the incision until it reaches the disc space 62. A second dilator 74, having an inner diameter that is slightly larger than the outer diameter of first dilator 72 is then passed over dilator 72 until it reaches disc space 62. A third dilator 76, a fourth dilator 78 and a fifth dilator 80, each having successively larger inner diameters, are then passed over the previous dilator into the disc space 62. As each dilator enters the disc space, it gradually and incrementally enlarges the height of disc space 62 until the disc space is at a desired height. The desired height does not have to be the maximum required height, since that height may be reached by the expandible disc spacer which will be inserted into the disc space. The number of dilators may of course vary, depending on the height of the disc space desired. The depth to which the dilators are inserted can be monitored in many known ways, such as by fluoroscopy, calibrations on the dilators, a combination of both, or other means.

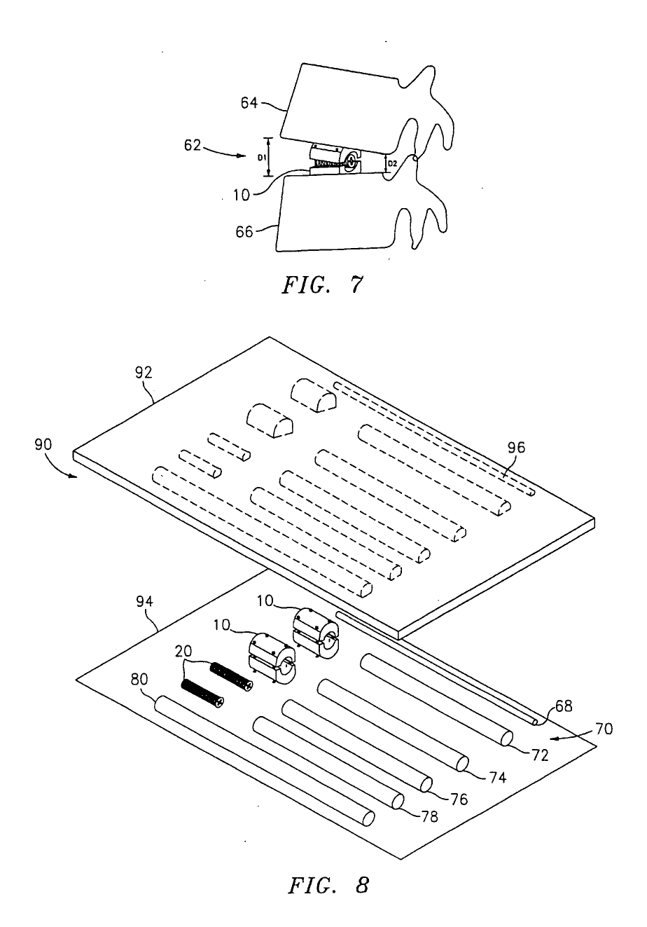

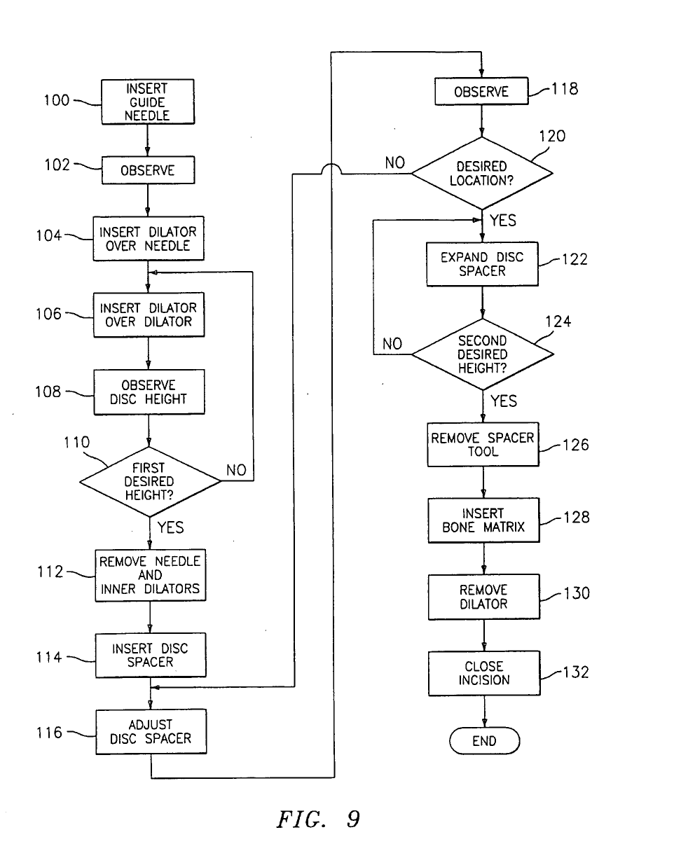

Referring to FIG. 6, once the dilators are in place, and the disc space 62 is at the desired height, the guide needle 68 and all the dilators, with exception of the outermost dilator 80, are removed. Expandible intervertebral disc spacer 10 is the passed through dilator 80 to the disc space 62 by an insertion tool 82. The position of disc spacer 10 is confirmed under fluoroscopy, and either tool 82 or another tool inserted through dilator 80 is used to tighten screw 20. Disc spacer 10 is positioned so that the first end of spacer 10, having the larger diameter 30 of tapered bore 18, is positioned posteriorly, while the second end having smaller diameter 32 of bore 18 is positioned anteriorly. As seen in FIG. 7, when the screw 20 is tightened, the second end, on the anterior side of the spine opens a distance D2, which is greater than distance D1, which is on the posterior side of the spine. This restores lordosis, or the natural curvature of the spine, particularly in the lumbar region, and relieves the intervertebral foramina and decompresses the nerve roots. Once the disc spacer 10 is in position, bone matrix is passed through the dilator 80 to encourage fusion, to fill the disc space with bone.

While the above procedure has been described for only one set of dilators, and for enlarging the disc space for placement of a disc spacer on one side of the disc space 62, it is understood that the procedure is performed on both sides of the disc space to raise the disc height evenly, and that two disc spacers 10 are inserted. After the bone matrix is inserted, the dilators are then removed and the procedure is complete.

FIG. 8 illustrates a kit for performing the percutaneous interbody fusion procedure of the present invention. Kit 90 comprises a package having top cover 92 and bottom cover 94, where top cover 92 is preferably formed of plastic having depressions or indentations 96 for holding the instruments packaged therein. Packaged in kit 90 are preferably at least two disc spacers 10, a corresponding number of screws 20, a plurality of dilators 70 and a guide needle 68. Kit 90 is preferably sterilized.

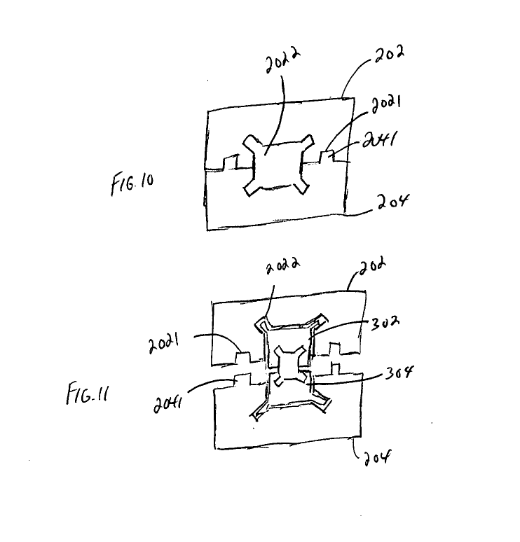

FIG. 9 is a flow chart of the method of the present invention. Following a percutaneous discectomy procedure, a guide needle is inserted through the incision at step 100 to the disc space between the vertebral bodies to be fused. The depth to which the guide needle is inserted is observed, preferably through fluoroscopy, in step 102. Once the guide needle is in place, a dilator having an inner diameter that is slightly larger than the outer diameter of the guide needle is passed over the guide needle to the disc space in step 104. The dilator increases the height of the disc space. In step 106, a second dilator is passed over the first dilator, where the second dilator has an inner diameter that is slightly larger than the outer diameter of the first dilator, to further increase or enlarge the disc space. At step 108, the height of the disc space is then observed, preferably through fluoroscopy, to see if it is at the desired height, at step 110. If not, the procedure returns to step 106 and another dilator, having an inner diameter slightly larger than the outer diameter of the previous dilator, is passed over the previous dilator to the disc space. If the disc space is at the desired height, the guide needle and all the dilators, with the exception of the outermost dilator, are removed at step 112. At step 114, an expandible intervertebral disc spacer is inserted through the dilator to the disc space. The position of the disc spacer is adjusted to a proper position at step 116, and then observed, preferably through fluoroscopy, at step 118. If it is determined at step 120 that the disc spacer is not at the correct location, the procedure returns to step 116. If the position is correct, the disc spacer is expanded to enlarge the disc space to a desired height at step 122. If it is determined at step 124 that the space is not at the desired height, the procedure returns to step 122. If the space is at the desired height, the tool is removed at step 126, and a bone matrix is passed down the dilator to the disc space in step 128. Once the bone matrix is in place, the dilator is removed at step 130, and the incision is closed at step 132, ending the procedure.

In addition, it is also contemplated that the method of the present invention may be used to perform surgical procedures for delivery of natural and artificial devices to the disc space after a discectomy has been performed. For example, prosthetic devices such as replacement discs, artificial or otherwise, may be implanted to replace the removed disc from the disc space. While replacement discs are known, current techniques suffer several disadvantages. In particular, the present disc replacement procedures require large incisions to access the disc space. In addition, once the replacement disc is in place, the patient must be immobilized for long periods of time to allow the disc to stabilize and for the patient to heal. Performing these procedures according to the method of the present invention decreases the recovery time.

Furthermore, it is contemplated that the expandible intervertebral disc spacer of the present invention can be constructed of a bio-absorbable material that will be absorbed by the body over time. The bio-absorbable material is of sufficient strength and provides adequate structural integrity to the spacer that as the spacer dissolves or is absorbed over a long period of time, the spacing between the vertebra is maintained to allow the replacement disc to stabilize, thereby preventing distortion or damage to the replacement disc following surgery. The patient gains mobility and recovery time is dramatically decreased, since the patient does not need to be immobilized for long periods of time. Collagen-based absorbable materials are contemplated, as well as any suitable bio-absorbable materials, including Vicryl, for the material of the spacer.

In accordance with an alternative embodiment, the pins may be constructed as pegs, along the seam where the two halves meet in order to connect the two halves of the disc spacer. The pegs fit into holes in the complementary seam, i.e., where the two halves meet. In this way, the pegs hold the two halves together initially, but when the expansion means, such as the screw, is put in between the two halves, the pegs are removed from the holes and the spacer expands to the desired height as discussed above.

In accordance with further aspects of the alternative embodiment, the expansion means itself may be constructed in the same way, two halves connected by pegs. Inserting a series of expanding means expands the spacer.

FIG. 10 shows a front view of a disc spacer in accordance with the alternative embodiment of the present invention. A top half 202 has holes 2021 in a bottom surface to accept pegs 2041 extending from a top surface of a bottom half 204. An aperture 2022 in the center of the spacer is shaped to accept an expander with pegs at its corners, the pegs extending from the corner of the expander at an angle. When the expander is inserted, recesses in the inner surface of each half fit over the pegs. These angled pins will keep its respective half attached thereto during expansion. The aperture 2022 preferably is tapered from a first end to a second end, such that the height of the aperture is greater at the first end than at the second end.

FIG. 11 shows a first expander with a top half 302 and bottom half 304 placed in the aperture 2022. The expander may be preferably tapered, as described above, to expand the spacer as it is inserted. In this way, the spacer is expanded to the desired height by inserting a series of expanders until the desired height is achieved. When the expander is tapered, aperture 2022 preferably has a constant height.

While the invention has been shown and described with reference to certain preferred embodiments, it will be understood by those skilled in the art that various changes and modifications in form and detail may be made therein without departing from the spirit and scope of the invention, as defined by the appended claims.

1. An expandible intervertebral disc spacer, comprising:
a split cylinder having a top half and a bottom half joined by a plurality of pins to permit movement of the top half with respect to the bottom half, an inner surface of the top half and an inner surface of the bottom half facing each other defining a tapered bore extending from a first end of the cylinder towards a second end of the cylinder; and
a piston screw insertable into the tapered bore to expand the cylinder by moving the top half away from the bottom half to cause the second end of the cylinder to expand a greater distance than the first end,
wherein the disc spacer is constructed of a material to be absorbed by a body.

2. An expandible intervertebral disc spacer, comprising:
a top half and a bottom half, an inner surface of the top half and an inner surface of the bottom half facing each other defining a tapered bore extending from a first end of the cylinder towards a second end of the cylinder; and
an expander insertable into the tapered bore to expand the cylinder by moving the top half away from the bottom half;
wherein the expander fits into recesses in the inner surface of the top half and the bottom half to cause the second end of the cylinder to expand a greater distance than the first end, and the disc spacer is constructed of a material to be absorbed by a body.

3. An expandible intervertebral disc spacer for implantation in a disc space to enlarge the height of the disc space and restore lordosis in a spine of a patient during interbody fusion procedures, comprising:
a top portion having an inner surface and an outer surface, and having a first end and a second end;
a bottom portion having an inner surface and an outer surface, and having a first end and a second end, the inner surface of the bottom portion facing the inner surface of the top portion;
a plurality of pins joining the top portion to the bottom portion to permit vertical movement of the top portion with respect to the bottom portion;
the inner surface of the top portion and the inner surface of the bottom portion defining a tapered bore having a diameter at a first end that is greater than a diameter at a second end; and
a rod for insertion into the tapered bore to expand a distance between the top portion and the bottom portion, the second end of the top portion moving a greater distance with respect to the second end of the bottom portion than the first end;
wherein the disc spacer is constructed of a material to be absorbed into the body.

4. The disc spacer of claim 3, wherein the tapered bore is threaded along at least a portion of its length.

5. The disc spacer of claim 4, wherein the rod is threaded.

6. The disc spacer of claim 3, wherein the top portion and bottom portion are constructed of bone material.

7. The disc spacer of claim 3, wherein the outer surface of the top portion and the outer surface of the bottom portion are scored to enhance adherence to vertebra of the spine.

8. An expandible intervertebral disc spacer, comprising:
a top half and a bottom half, an inner surface of the top half and an inner surface of the bottom half facing each other defining a bore extending from a first end of the cylinder towards a second end of the cylinder; and
a tapered expander insertable into the bore to expand the cylinder by moving the top half away from the bottom half;
wherein the expander fits into recesses in the inner surface of the top half and the bottom half to cause the second end of the cylinder to expand a greater distance than the first end, and the disc spacer is constructed of a material to be absorbed by a body.
